(19) United States
(12) Patent Application Publication
(10) Pub. No.: US 2016/0251679 A1
(43) Pub. Date: Sep. 1, 2016

(54) REGULATORY NON-CODING RNAS AS DETERMINANTS OF MALE STERILITY IN GRASSES AND OTHER MONOCOTYLEDONOUS PLANTS

(71) Applicants: Blake MEYERS, Wilmington, DE (US); Jixian ZHAI, Los Angeles, CA (US); Virginia WALBOT, Palo Alto, CA (US)

(72) Inventors: Blake Meyers, Wilmington, DE (US); Jixian Zhai, Los Angeles, CA (US); Virginia Walbot, Palo Alto, CA (US)

(73) Assignee: University of Delaware, Newark, DE (US)

(21) Appl. No.: 15/028,447

(22) PCT Filed: Oct. 10, 2014
(86) PCT No.: PCT/US2014/060081

§ 371 Date: Apr. 11, 2016

Related U.S. Application Data
(60) Provisional application No. 61/889,587, filed on Oct. 11, 2013.

(51) Int. Cl. C12N 15/82; A01H 1/02

(52) U.S. Cl. CPC C12N 15/8289; C12N 15/8218; A01H 1/02

(57) ABSTRACT

A method for controlling male fertility of a plant comprises regulating a biological activity of a phasiRNA in a male reproductive organ of the plant. The phasiRNA is selected from the group consisting of 21-nt phasiRNAs and 24-nt phasiRNAs. The male fertility of the plant is thereby increased or decreased. Preferably, the plant is a monocotyledon and becomes male sterile. The resulting male sterile plant and its uses are also provided.

### CROSS-REFERENCE TO RELATED APPLICATION

This application claims the benefit of U.S. Provisional Application No. 61/889,587, filed Oct. 11, 2013, the contents of which are incorporated herein in their entireties for all purposes.

### FIELD OF THE INVENTION

The invention relates generally to plant genetic engineering, especially the use of phased small RNAs (phasiRNAs) for controlling male fertility in plants.

### BACKGROUND OF THE INVENTION

Diverse small RNAs exist in male reproductive cells of animals and plants. In animals, PIWI proteins and their interacting piRNAs are required for spermatogenesis; mutants defective for the PIWI-encoding genes fail to produce mature sperm. While most Drosophila piRNAs are repeat-derived and silence transposable elements (TEs), mammalian piRNAs predominantly map to unique intergenic regions and have unclear but essential roles during gonad development. Based on their expression timing, different sizes, and distinctive PIWI partners, mammalian piRNAs are further classified as pre-pachytene or pachytene. Given the continuum of developmental stages in the testes, the pre-pachytene class is characteristic of gonads in which no cells have reached pachytene while the pachytene-associated small RNAs are characteristic of gonads in which the most advanced germ line cells have reached this meiotic stage and all prior stages are also present in the more immature zone of the gonad.

In flowering plants, the anther is equivalent to the mammalian testes in that it consists of multiple somatic cell types required to support the pre-meiotic, meiotic, and post-meiotic haploid cells. In contrast to the continuum of mammalian gonads, however, an entire anther progresses through sequential developmental landmarks, and in maize, meiosis is synchronous within the organ. A second major difference between plants and animals is that the haploid meiotic products of plants are microspores, which undergo mitotic divisions to produce the three-celled gametophyte. Two of the gametophytic cells are sperm—later involved in double fertilization—and the third cell is a metabolically active, haploid vegetative cell. Like their mammalian counterparts, the plant germ line also contains repeat and non-repeat derived small RNAs. In Arabidopsis pollen, TE-derived small interfering RNAs (siRNAs) expressed in the vegetative nuclei reinforce silencing after transfer to sperm nuclei. Additionally, rice inflorescences produce 21- and 24-nt phased, secondary siRNAs (phasiRNAs) from non-repeat regions.

A key step in the production of many plant secondary siRNAs is cleavage of their precursors by a 22-nt microRNA (miRNA). In the case of grass phasiRNAs, their mRNA precursors—“PHAS” transcripts—are transcribed by RNA polymerase II, capped and polyadenylated. These long non-coding precursor transcripts are internally cleaved, guided by 22-nt miR2118 to generate the 21-nt phasiRNAs or by miR2275 for the 24-nt phasiRNA (FIG. 1A). RNA-Dependent RNA Polymerase 6 (RDR6) recognizes the cleaved, uncapped 3′ fragments of these transcripts and synthesizes a second strand, forming double stranded RNA. Subsequent processing by Dicer-Like 4 (DCL4) and Dicer-Like 5 (DCL5) generates 21- and 24-nt phasiRNAs, respectively. Both dicers exhibit sequential slicing activity, starting precisely at the 11th nucleotide of the miRNA binding site. This activity generates populations of regularly spaced, phased siRNAs from each PHAS precursor.

Although plants lack PIWI-clade ARGONAUTEs that bind piRNAs, the plant Argonaute (AGO) family has diversified extensively, and there are plant-specific AGO proteins. Meiosis Arrested At Leptotene 1 (MEL1), a rice homolog of Arabidopsis AGO5, mainly localizes to the cytoplasm of pre-meiotic cells. Recently MEL1 was shown to selectively bind 21-nt phasiRNAs. mel1 loss of function mutants have abnormal tapetum and aberrant pollen mother cells (PMC, the final differentiated state prior to the start of meiosis) that arrest in early meiosis, suggesting that 21-nt phasiRNAs are crucial for male fertility.

Male sterile plants are useful in producing desirable hybrid seeds to develop plant varieties and improve crop yield. There remains a need for methods of controlling male fertility effectively in plants.

### SUMMARY OF THE INVENTION

The present invention provides a method for controlling male fertility of a plant. The method comprises regulating a biological activity of a phasiRNA in a male reproductive organ of the plant. The phasiRNA is selected from the group consisting of 21-nt phasiRNAs and 24-nt phasiRNAs. The male fertility of the plant is thereby increased or decreased. The plant is preferably a monocotyledon, for example, maize.

The method may further comprise regulating the expression of the phasiRNA in cells of the male reproductive organ. The biological activity of the phasiRNA is thereby increased or decreased.

The method may further comprise regulating the expression in cells of the male reproductive organ of an mRNA precursor (PHAS) of the phasiRNA, a 22-nt microRNA (miRNA) capable of cleaving the PHAS to make the phasiRNA, or a facilitating protein capable of regulating the expression of the phasiRNA in the plant. The expression of the phasiRNA is thereby increased or decreased.

The method may further comprise introducing into cells of the male reproductive organ an effective amount of a nucleic acid molecule that is antagonistic to the phasiRNA, the mRNA precursor (PHAS), or the 22-nt microRNA (miRNA). The expression of the phasiRNA, the mRNA precursor (PHAS), or the 22-nt microRNA (miRNA) is thereby increased or decreased.

The method may further comprise regulating the expression of RNA-Dependent RNA Polymerase 6 (RDR6) in cells of the male reproductive organ. The expression of the mRNA precursor (PHAS) is thereby increased or decreased.

In some embodiments, the phasiRNA is a 21-nt phasiRNA, the 22-nt miRNA is miR2118, and the facilitating protein is selected from the group consisting a dicer protein and an Argonaute (AGO) protein. The dicer protein may be DICER-LIKE4 (DCL4). The Argonaute (AGO) protein may be an AGO5-related protein. For example, the plant may be rice and the AGO5-related protein may be Meiosis Arrested At Leptotene 1 (MEL1).

In other embodiments, the phasiRNA is a 24-nt phasiRNA, the 22-nt miRNA is miR2275, and the facilitating protein is selected from the group consisting a dicer protein and an Argonaute (AGO) protein. The dicer protein may be DICER-LIKE5 (DCL5). The Argonaute (AGO) protein may be an AGO18 protein. For example, the plant may be maize and the AGO18 protein may be selected from the group consisting of GRMZM2G105250 and GRMZM2G457370.

In some preferred embodiments, the plant is male sterile. A male sterile plant obtained in accordance with the method of the present invention is also provided. A plant cell or tissue obtained from the male sterile plant is further provided.

The present invention also provides a method for producing a hybrid seed. The method comprises crossing the male sterile plant of the present invention with another plant. A hybrid seed is thereby produced. The hybrid seed produced in accordance with this method is further provided.

### BRIEF DESCRIPTION OF THE DRAWINGS

FIGS. 1A-B illustrate genome-wide identification of 21-nt and 24-nt phasiRNAs loci in maize. (A) PhasiRNA biogenesis pathways, 21-nt phasiRNAs at left and 24-nt phasiRNAs at right, result in loci with characteristic phased patterns. (B) Distribution of 21-PHAS (left side of the chromosome) and 24-PHAS (right side of the chromosome) loci on 10 maize chromosomes. Loci within 500,000 bp are clustered together; the number adjacent to each bar represents the number of loci in that particular cluster.

FIGS. 2A-C show 21-nt pre-meiotic and 24-nt meiotic phasiRNAs are developmentally regulated. (A) Anthers at ten different lengths (developmental stages) were analyzed plus pollen. Above, a schematic of cell patterns in a single lobe of the anther; cell types indicated in different shades of grey. (B) Heat maps depicting the abundances of 21-nt pre-meiotic phasiRNAs from 463 loci (left panel) and 24-nt meiotic phasiRNAs from 176 loci (right panel) at each stage. Hierarchical clustering was based on similarity of expression pattern. Left panel: solid bubbles represent the total abundance of phasiRNAs; pie-charts represent the proportion of 21-nt phasiRNAs from all 21-nt sRNAs at each stage; striped bubbles represent miR2118 abundances (trigger of pre-meiotic phasiRNA). Right panel: the solid and striped bubbles represent 24-nt phasiRNA and miR2275 abundances, respectively; pie-charts represent 24-nt phasiRNAs from all 24-nt sRNAs. Controls at far right: left side represents TAS3-derived ta-siRNAs, and right represents all TE-associated siRNAs mapped to the first 100 Mb of maize chromosome 1 (as a proxy for the whole genome). (C) Quantification of 21-PHAS and 24-PHAS precursor transcripts by RNA-seq.

FIGS. 3A-D show impact of maize male-sterile mutants on the accumulation of miRNA triggers, PHAS precursors and phasiRNAs. (A) Illustration of cell layer organization in fertile, ocl4, msca1, mac1, ms23 and ameiotic1 anther lobes at 0.7 mm. Color key as in FIG. 2A. (B) Quantification of 21-phasiRNAs and miR2118 in fertile, ocl4, msca1, mac1, ms23 and am1-489 mutants; colors as in FIG. 2B. All dots with the same shade were normalized together (and across the genotypes), to permit comparison across all time points and genotypes. (n/a, not available.) (C) Quantification of 24-phasiRNAs and miR2275 triggers in fertile and mutant anthers. (D) TAS3-derived ta-siRNAs and TE-derived siRNAs are shown as control sRNA.

FIG. 4 shows impact of maize male-sterile mutants on the accumulation of PHAS precursors profiled by RNA-seq. Quantification of 21-PHAS and 24-PHAS in fertile, ocl4, mac1 and ms23; the 21- and 24-nt PHAS precursor abundances are indicated as per the legend below the figure.

FIGS. 5A-H show localization of phasiRNA biogenesis components in developing anthers. Small RNA in situ hybridization with a probe for A) miR2118, D) pre-meiotic phasiRNA from 21-PHAS_NO142, E) miR2275 and H) meiotic phasiRNA from 24-PHAS_NO132. Regular mRNA in situ hybridization with probes for B) 21-PHAS_NO142, C) DCL4, F) 24-PHAS_NO132, and G) DCL5. Scale bar=20 μm, for all images.

FIG. 6 is a cartoon illustration of proposed movement of phasiRNAs. Pre-meiotic phasiRNAs are generated in the epidermis and transfer to the sub-epidermal cells (A) while meiotic phasiRNAs move from the tapetum to PMC (B) to perform their functions.

FIGS. 7A-B show the abundances in transcripts per 10 million of mRNAs for genes encoding Argonaute (AGO) proteins during meiosis in maize. Panel A lists the abundances as numerical values, which shading indicative of higher abundances, and panel B displays these values as a line graph.

### DETAILED DESCRIPTION OF THE INVENTION

Maize anthers, the male reproductive floral organs, express two classes of phased small RNAs (phasiRNAs). PhasiRNA precursors are transcribed by RNA polymerase II and map to low copy, intergenic regions similar to piRNAs in mammalian testis. From ten sequential cohorts of staged maize anthers plus mature pollen, it has been found that 21-nt phased siRNAs from 463 loci appear abruptly after germinal and initial somatic cell fate specification and then diminish, while 24-nt phasiRNAs from 176 loci coordinately accumulate during meiosis and persist as anther somatic cells mature and haploid gametophytes differentiate into pollen. Male-sterile ocl4 anthers defective in epidermal signaling lack 21-phasiRNAs. Male-sterile mutants with subepidermal defects—mac1 (excess meiocytes), ms23 (defective pre-tapetal cells), and msca1 (no normal soma or meiocytes)—lack 24-phasiRNAs. Ameiotic1 mutants (normal soma, no meiosis) accumulate both 21- and 24-phasiRNAs, ruling out meiotic cells as a source or regulator of phasiRNA biogenesis. By in situ hybridization, miR2118 triggers of 21-phasiRNA biogenesis localize to epidermis, however, 21-PHAS precursors and phasiRNAs are abundant subepidermally. The miR2775 trigger, 24-PHAS precursors, and 24-phasiRNAs all accumulate preferentially in tapetum. Each phasiRNA type has been found to exhibit independent spatiotemporal regulation with 21-nt phasiRNAs dependent on epidermal and 24-phasiRNAs dependent on tapetal cell differentiation. Maize phasiRNAs and mammalian PIWI-interacting RNAs (piRNAs) illustrate convergent evolution of small RNAs to support male reproduction.

The present invention is based on the discovery of the role and the use of short or long non-coding RNAs in the development of male reproductive organs in plants. In particular, novel functions of two classes of phased, secondary small interfering RNAs (phasiRNAs) in male reproduction have been discovered, and alteration of the function or biogenesis of these phasiRNAs result in a change to male fertility, even male sterility. This male sterility can be used as a genetic tool to promote outcrossing in plants, for example, grasses or non-grasses monocots. Such outcrossing is fundamental to the reproduction of hybrid seeds, which often exhibit hybrid vigor.

The objective of the present invention includes providing a genetic mechanism to control male fertility and sterility, and to facilitate the production of hybrid seeds. There may be secondary roles in the improvement of male fertility under adverse environmental conditions. Also, it may be possible to target these RNAs using exogenously applied factors to trigger male sterility using a non-genetic method. This could include RNA or DNA molecules that are antagonistic to the non-coding RNAs or the use of microorganisms, including fungi, to deliver proteins, RNA, or DNA to disrupt or enhance the phasiRNA production pathways.

The present invention provides a method for controlling male fertility of a plant. The method comprises regulating a biological activity of a phasiRNA in a male reproductive organ of the plant. The male fertility of the plant is increased or decreased.

The term “male fertility” used herein refers to the failure of a plant to produce functional anthers, pollen, or male gametes. The term “male reproductive organ” used herein refers to a male reproductive floral organ, for example, maize anthers. The plant male fertility may be increased or decreased by at least about 10%, 20%, 30%, 40%, 50%, 60%, 70%, 80%, 90%, 95% or 99%. The plant male fertility may be determined by conventional techniques known in the art.

The term “phased small RNA” or “phasiRNA” used herein refers to a double-stranded ribonucleic acid (dsRNA) molecule from eukaryotic cells that interferes with the expression of a specific gene with a nucleotide sequence complementary to one strand of the dsRNA. The phasiRNA may act in trans as tasiRNA or in cis as casiRNA, where trans indicates that the target of the phasiRNA is produced from the mRNA of a different gene than the phasiRNA, and cis indicates that the target of the phasiRNA is the mRNA of the same gene that produces the phasiRNA. The phasiRNA may have 20 to 25 nucleotides (nt) in length, preferably 21 nt or 24 nt. The phasiRNA may be a naturally occurring phasiRNA, or artificially synthesized having a sequence at least about 70%, 80%, 90%, 95% or 99%, preferably at least about 80%, more preferably about 100%, identical to a naturally occurring phasiRNA. The phasiRNA may be generated from an mRNA precursor (PHAS). Table 1 provides the positions and coordinate in the maize genome sequence (“version 2”) of the loci that produce the 21- and 24-phasiRNAs, sorted by abundance from greatest to least. Each of these loci may generate more than 20 phasiRNAs. The units of the “100,000” are transcripts per 10 million reads, and the abundances in this table are the sum of abundance of all phasiRNAs of either 21 or 24 nt from each locus. The phasiRNA may be generated in a unit of either 21 or 24 nt from within these loci. The phasiRNA may have a sequence at least about 50%, 60%, 70%, 80%, 90%, 95% or 99%, preferably at least about 80%, more preferably at least 95%, most preferably about 100%, identical a stretch of either 21 or 24 nt within any of these loci.

The term “biological activity” used herein refers to any activity of a phasiRNA relating to plant male fertility. For example, the biological activity of a 21-nt phasiRNA may be related to post-transcriptional control of RNA targets. Exemplary RNA targets include the set of all parental mRNAs, or a subset thereof. The biological activity of a 24-nt phasiRNA may be related to directing chromatin modifications at its target site. For example, the target site may be DNA sequences on the chromosomes, or may be RNAs transcribed by RNA polymerases II, IV, or V.

The plant may be a monocotyledon. The monocotyledon may be a grass or a non-grass. Examples of grasses include maize, rice, wheat, barley, sorghum, switchgrass and sugarcane. Examples of non-grasses include asparagus, banana and palm. Preferably, the plant is rice or maize. More preferably, the plant is maize.

The method may further comprise regulating the expression of the phasiRNA in cells of the male reproductive organ. The biological activity of the phasiRNA is thereby increased or decreased, for example, by at least about 10%, 20%, 30%, 40%, 50%, 60%, 70%, 80%, 90%, 95% or 99%. The expression of the phasiRNA may be detected by conventional techniques known in the art, and may be up or down regulated in some or all of the cells of the male reproductive organ.

The method may further comprise regulating the expression in cells of the male reproductive organ of an mRNA precursor (PHAS) of the phasiRNA, a 22-nt microRNA (miRNA) capable of cleaving the PHAS to make the phasiRNA, or a facilitating protein capable of regulating the expression of the phasiRNA. The expression of the phasiRNA is thereby increased or decreased, for example, by at least about 10%, 20%, 30%, 40%, 50%, 60%, 70%, 80%, 90%, 95% or 99%. The expression of the PHAS, the 22-nt miRNA, or the facilitating protein may be detected by conventional techniques known in the art, and may be up or down regulated in some or all of the cells of the male reproductive organ.

The method may further comprise introducing into cells of the male reproductive organ an effective amount of a nucleic acid molecule that is antagonistic to the phasiRNA, the mRNA precursor (PHAS), or the 22-nt microRNA (miRNA). The expression of the phasiRNA, the mRNA precursor (PHAS), or the 22-nt microRNA (miRNA) is thereby increased or decreased. The nucleic acid molecule may be introduced into the cells using conventional techniques known in the art. The introduction may be transient or permanent, preferably permanently. The nucleic acid molecule may be introduced into the cells over a period of hours, days, weeks or months. It may also be introduced once, twice, or more times. The effective amount of the nucleic acid molecule may vary depending on various factors, for example, the sequence of the nucleic acid molecule, the physical characteristics of the cells, the sequence of the phasiRNA, the PHAS or the 22-nt miRNA, and the means of introducing the nucleic acid molecule into the cells. A specific amount of the nucleic acid molecule to be introduced may be determined by one using conventional techniques known in the art.

The method may further comprise regulating the expression of RNA-Dependent RNA Polymerase 6 (RDR6) in cells of the male reproductive organ. The expression of the PHAS is thereby increased or decreased, for example, by at least about 10%, 20%, 30%, 40%, 50%, 60%, 70%, 80%, 90%, 95% or 99%. The expression of RDR6 may be detected by conventional techniques known in the art, and may be up or down regulated in some or all of the cells of the male reproductive organ.

In some embodiments, the phasiRNA is a 21-nt phasiRNA, the 22-nt miRNA is miR2118, and the facilitating protein is selected from the group consisting a dicer protein and an Argonaute (AGO) protein. The dicer protein may be DICER-LIKE4 (DCL4). The AGO protein may be an AGO5-related protein. The AGO5-related protein may be Meiosis Arrested At Leptotene 1 (MEL1).

In other embodiments, the phasiRNA is a 24-nt phasiRNA, the 22-nt miRNA is miR2275, and the facilitating protein is selected from the group consisting a dicer protein and an Argonaute (AGO) protein. The dicer protein may be DICER-LIKE5 (DCL5), also known as DCL3b. The AGO protein may be an AGO18 protein. In maize, the AGO18 protein may be selected from the group consisting of GRMZM2G105250 and GRMZM2G457370.

In some preferred embodiments, the plant becomes male sterile. The resulting male sterile plant as well as its cells or tissues are also provided.

According to another aspect of the present invention, a method for producing a hybrid seed is provided. The method comprises crossing the male sterile plant of the present invention with another plant, which preferably belongs to the same genus, more preferably the same species, as the male sterile plant. For example, the male sterile plant and the plant with which the male sterile plant is crossed are both rice or maize. The resulting hybrid seed is also provided.

The term “about” as used herein when referring to a measurable value such as an amount, a percentage, and the like, is meant to encompass variations of ±20% or ±10%, preferably ±5%, more preferably ±1% from the special value, as such variations are appropriate to perform the disclosed methods.

The following examples are provided to describe exemplary aspects of the invention in greater detail. They are intended to illustrate, not to limit, the invention.

### Example 1

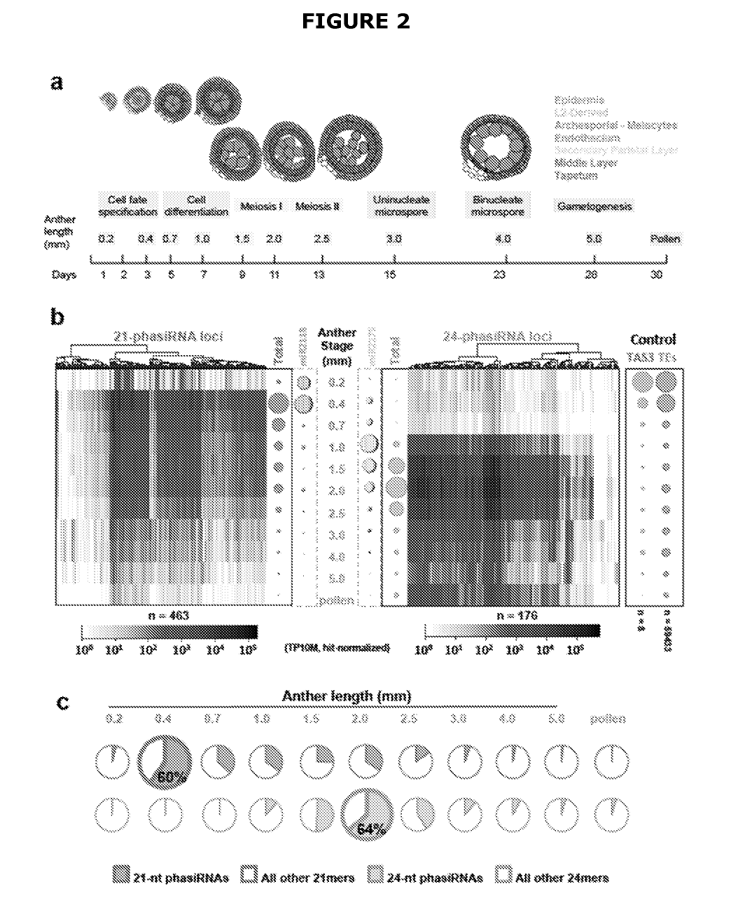

As a monoecious plant with large cohorts of synchronously developing flowers, maize (Zea mays) is particularly useful for studying male reproduction; anthers are readily dissected and staged using length as a proxy for developmental events (FIG. 2A). We utilized this developmental regularity to characterize the spatiotemporal patterns of phasiRNA accumulation. Small RNA-seq and RNA-seq were applied to 11 sequential wild type (fertile) stages, ranging from the initial step of cell fate specification in anther primordia through pollen production. We demonstrated that both phasiRNAs and their precursor transcripts show striking spatiotemporal regulation.

### Results

Temporal Regulation of Pre-Meiotic and Meiotic phasiRNAs

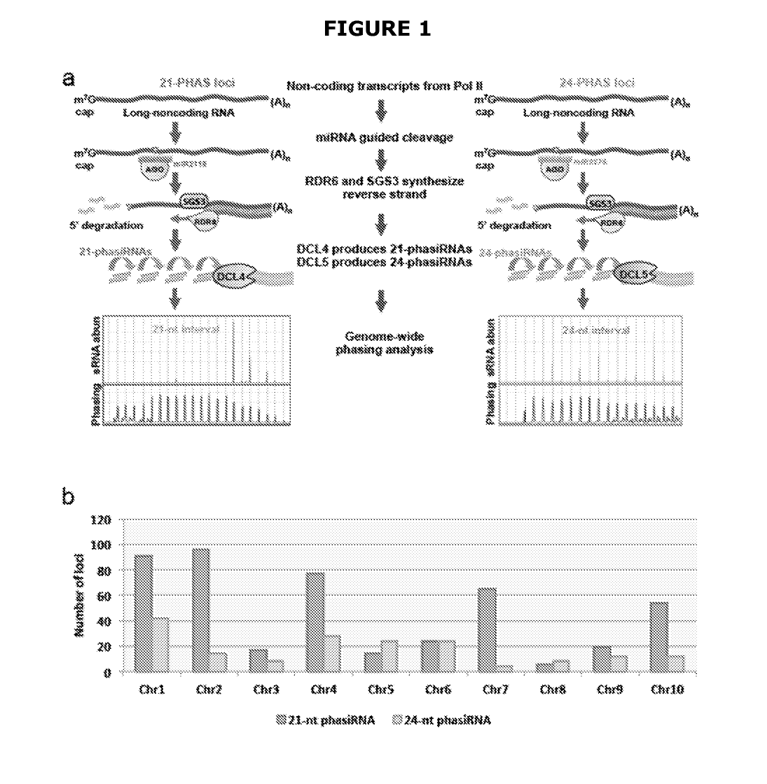

To explore the dynamics of small RNA populations in male reproductive organs of maize, 32 small RNA (sRNA) libraries from 11 sequential stages of W23 fertile anthers were sequenced deeply to allow accurate and sensitive identification of phasiRNAs. The phasiRNAs were then mapped to the genome by computational, genome-wide scans, identifying 463 21-PHAS and 176 24-PHAS loci; both classes of loci are distributed on all 10 maize chromosomes (FIG. 1B). These loci correspond to unique or low copy genomic regions. This distinguishes the 24-nt phasiRNAs from plant DCL3-dependent, 24-nt heterochromatic siRNAs (hc-siRNAs), which are largely derived from repetitive elements, primarily TEs.

Both 21-nt and 24-nt phasiRNAs exhibit striking temporal regulation (FIG. 2B) distinct from the timing of either TAS3-derived 21-nt trans-acting siRNAs (ta-siRNAs) or 24-nt hc-siRNAs derived from TE (FIG. 2B). Few phasiRNAs were observed at 0.2 mm when germinal and initial somatic fate-setting starts from pluripotent stem cells. After 24 h at the 0.4 mm stage when pre-meiotic archesporial (AR) and three somatic cell types—epidermis, endothecium, bipotent secondary parietal layer (SPL)—exist, 21-nt phasiRNAs peak in quantity and diversity, comprising 60% of all 21-nt RNAs (FIG. 2B). Most 21-nt phasiRNAs are present for approximately one week (0.4 mm to 2.0 mm stages), but decline steadily from 0.7 mm when all SPL cells have divided, producing middle layer and tapetal daughter cells. In contrast, most 24-nt phasiRNAs are undetectable until 1.0 mm, when all cell types are specified and the post-mitotic AR start meiotic preparation as PMC (FIG. 2B). The 24-nt phasiRNAs peak from 1.5 to 2.0 mm, coincident with meiotic progression through prophase I to metaphase I, and when somatic cells continue differentiating for post-meiotic supporting roles; at this peak, 24-nt phasiRNAs reached 64% of all 24-nt RNAs (FIG. 2B). Most 24-nt phasiRNAs are present when meiosis finishes (2.5 mm), then decline in abundance but remain detectable in mature pollen, two weeks later. PhasiRNA dynamics were validated with three biological replicates and by RNA hybridization. Based on their expression timing, we named the two size classes pre-meiotic (21-nt) and meiotic (24-nt) phasiRNAs to highlight the parallels with mammalian gonad piRNAs.

PhasiRNA synthesis requires both miRNAs and PHAS precursors (FIG. 1A). miR2118 family members were abundant at 0.2 mm, peaked at 0.4 mm, then vanished by 0.7 mm. In contrast, miR2275 family members peaked at 1.0 mm (FIG. 2B). Both miRNA families accumulate to their peak prior to that of the corresponding phasiRNA burst. RNA-seq from all eleven anther stages demonstrated that 21-PHAS precursor transcripts are highly expressed from 0.4 to 1.0 mm, while 24-PHAS transcripts peak in 1.5 mm anthers (FIG. 2C). Collectively, all three components exhibit tight timing in developing anthers: miRNA triggers precede coordinate deployment of PHAS precursors and their phasiRNA products.

Epidermis is Necessary and Sufficient for Pre-Meiotic phasiRNA Biogenesis

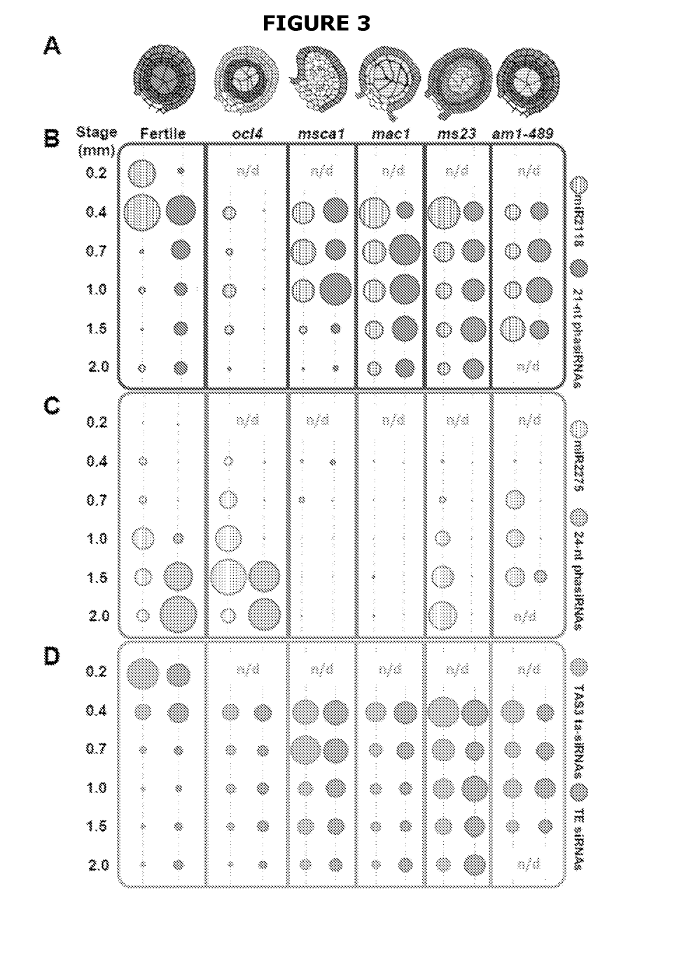

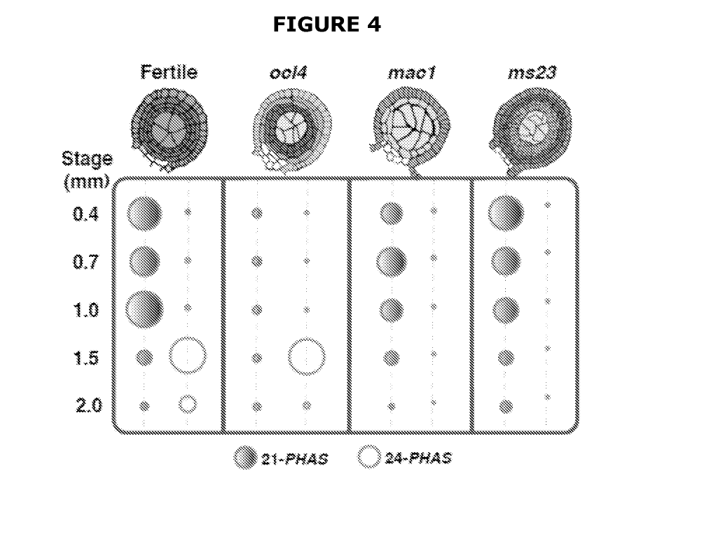

To gain insight into cell type contributions to phasiRNA production, RNAs were analyzed from developmental mutants defective in specific anther cell types (FIG. 3A). OCL4 is an epidermal-specific transcription factor repressing periclinal divisions in the adjacent subepidermal endothecial cells, presumably through a mobile signal. ocl4 anthers lack all 21-nt pre-meiotic phasiRNAs despite containing reduced but robust levels of the miR2118 trigger (FIG. 3B). Because ocl4 accumulates other RDR6/DCL4 products such as TAS3 ta-siRNAs (FIG. 3D), the defect could be in the production of 21-PHAS precursors. Indeed, RNA-seq of 0.4 to 2.0 mm anthers showed that ocl4 lacks 21-PHAS transcripts (FIG. 4). ocl4 has nearly normal timing and abundances of miR2275, 24-PHAS precursors, and 24-nt meiotic phasiRNAs (FIGS. 3C and 4), indicating their independence from both epidermal regulation and the pre-meiotic phasiRNA pathway. Furthermore, in msca1, in which mutant organs retain anther shape but no anther lobe cell types exist except the epidermis, there were near-normal levels of pre-meiotic phasiRNAs, with prolonged, elevated levels of miR2118 (FIG. 3B). We conclude, based on ocl4 and msca1 analysis, that a differentiated anther epidermis is necessary and sufficient for pre-meiotic phasiRNA biogenesis.

A Tapetal Layer, but not Meiocytes, is Required for Producing Meiotic phasiRNAs

To further explore cell requirements, additional male-sterile mutants were analyzed. mac1 mutants have excessive AR cells that mature and start meiosis, but typically the mutant anthers have only a single, undifferentiated sub-epidermal cell population; ms23 mutants have a normal endothecium and middle layer but pre-tapetal cells divide periclinally, forming an abnormal, undifferentiated bilayer (FIG. 3A). The mac1 and ms23 anthers as well as msca1 lack meiotic phasiRNAs (FIG. 3C), suggesting that a specified tapetal layer is required for meiotic phasiRNAs. Interestingly, while mac1 lacks both miR2275 and 24-PHAS transcripts, ms23 retains nearly normal miR2275 levels but is missing 24-PHAS precursors (FIG. 4). Together with the conclusions from analysis of ocl4, these data suggest that in both pre-meiotic and meiotic phasiRNA pathways, the production of the miRNA triggers and the PHAS precursors are separately regulated.

To test whether normal meiocytes are required for phasiRNAs production, we sequenced anther sRNAs from ameiotic1 (am1), in which somatic lobe cells are normal but PMC or meiocytes are defective. Two am1 alleles, am1-489 (PMCs conduct mitosis instead of meiosis) and am1-praI (meiocytes arrest in prophase I), have pre-meiotic and meiotic phasiRNAs (FIGS. 3B-C). Additionally, RDR6 transcripts, a shared biogenesis factor for both phasiRNA types, are not detected in fertile germinal cells by in situ hybridization. Therefore, phasiRNA production does not require normally differentiated germinal cells.

Spatial Distribution of phasiRNA Pathway Components

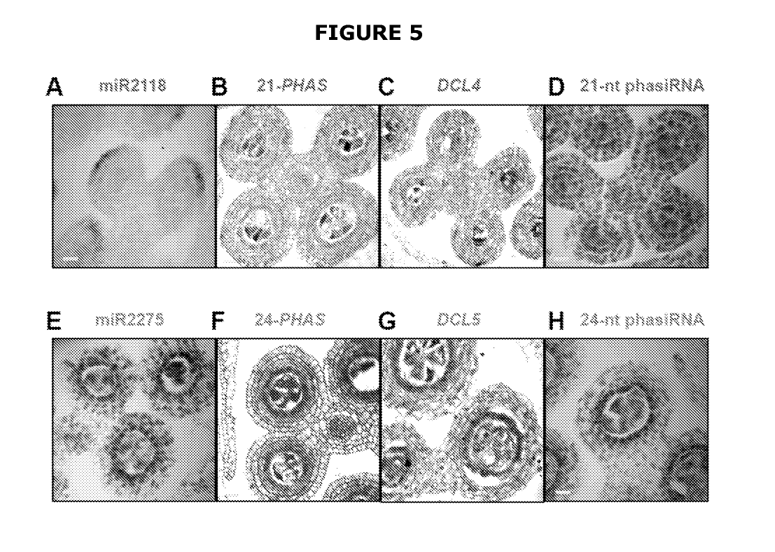

To further examine the spatial distribution of phasiRNAs, in situ localizations were performed on fertile anthers. miR2118 accumulated in a distal epidermal arc at 0.4 mm (FIG. 5A), precisely where the OCL4 transcription factor is expressed. 21-PHAS precursors and DCL4 transcripts are slightly enriched in AR cells while 21-nt pre-meiotic phasiRNAs localized to subepidermal cells (FIGS. 5B-D). Key biogenesis components for the 24-nt meiotic phasiRNAs were enriched in the tapetum and to a lesser extent in the pre-meiotic AR, PMC, and meiocytes (FIGS. 5E-5H). Therefore, the in situ results further support the distinct niches of the epidermis and tapetum in phasiRNA biogenesis. In addition, the separation of components required for biogenesis of pre-meiotic phasiRNAs suggests movement of one or more factors. The later-appearing meiotic phasiRNAs require tapetal differentiation, where biogenesis components co-localize. Tapetal cells are crucial for anther function; they secrete nutrients to support meiosis and later build the outer pollen coat. Because AR and PMC contain meiotic phasiRNAs, we speculate that these RNAs may be an additional type of “cargo” that tapetal cells supply to developing meiocytes.

### PhasiRNAs Lack Sequence Complementarity to TEs

Plant miRNAs and ta-siRNAs trigger target mRNA cleavage; such cleaved sites can be validated in bulk using Parallel Analysis of RNA Ends (PARE). To investigate possible targets of phasiRNAs, we constructed PARE libraries from several anther stages and mature pollen. Sequencing confirmed cleavage of 21- and 24-PHAS precursors by miR2118 and miR2275, respectively. From the ˜9 million predicted phasiRNA-target pairs of the 1,000 most abundant pre-meiotic phasiRNAs, fewer than 1% showed a PARE signal. These results are consistent with an earlier conclusion from rice that 21-nt pre-meiotic phasiRNAs lack obvious targets. Furthermore, an absence of pre-meiotic or meiotic phasiRNAs in ocl4, mac1 and ms23 does not result in TE transcript accumulation. The massive complexity of phasiRNAs and the lack of obvious target or association with transposons suggest that phasiRNAs function distinctively from miRNAs, ta-siRNAs, or hc-siRNAs.

### Discussion

Using small RNA-seq and RNA-seq, we demonstrated that pre-meiotic and meiotic phasiRNAs accumulate to high levels in maize anthers. Their accumulation is coordinated temporally with the expression of the precursor transcripts and preceded by accumulation of the corresponding miRNA triggers. Analysis of five male-sterile mutants defective in anther development showed that the two types of phasiRNAs are regulated independently. A normal epidermis is necessary and sufficient for pre-meiotic phasiRNA biogenesis, while the meiotic phasiRNAs require normal tapetal formation. In situ hybridization identified the localization of PHAS precursors, miRNA triggers and phasiRNAs, and confirmed the importance of epidermis in pre-meiotic phasiRNA and tapetum in meiotic phasiRNA production.

### Binding Partners of PhasiRNAs

Although plants lack PIWI-clade ARGONAUTEs that bind piRNAs, the plant AGO family has diversified extensively with 10 AGO members in Arabidopsis, 17 in maize, and 19 in rice. Some AGO members are specifically expressed in flowers and are further enriched in either somatic or germinal cells of anthers. Presumably, this AGO expansion reflects a functional diversification of plant small RNAs for roles specific to anther developmental stages and cell types.

Recently it has been shown that the rice Argonaute MEL1 binds 21-nt phasiRNAs. The closest homolog of MEL1 in maize is AGO5c. Maize AGO5c is highly expressed in 0.7 mm anthers, after pre-meiotic phasiRNAs peak. Therefore, AGO5c is likely the binding partner of pre-meiotic phasiRNAs in maize. The binding partner of meiotic phasiRNAs has not yet been reported. Based on transcriptional analysis of laser-microdissected cell types, plus the RNA-seq and microarray profiling of different anther stages, we found that the expression profile of maize AGO18b matches the expression timing of meiotic phasiRNAs. Both AGO18b transcripts and proteins are enriched in the tapetal and meiotic cells. Because it mirrors the distribution and timing of meiotic phasiRNAs, and like them is a recently evolved gene absent in dicots, AGO18b is strongly implicated as the partner of the meiotic phasiRNAs.

Proposed Functions of phasiRNAs

Although phasiRNAs lack sequence complementarity to TEs, they may have the capacity for genome surveillance of reproductive somatic and/or germinal cell transcripts, similar to what has been reported for Caenorhabditis elegans piRNAs (also known as 21U-RNAs). In flowering plants, TE silencing pathways are heavily redundant to ensure genome integrity. For example, even in the maize rdr2/mop1 mutant in which 24-nt hc-siRNAs are missing, there are only modest changes in TE expression. Given their large genomes containing many repetitive elements, the grasses may have evolved additional pathways operating through the phasiRNAs to regulate the TEs. It is also plausible that the phasiRNAs guard the anther somatic and germinal cell genomes against attack by pathogens such as viruses, fungi, or oomycetes, or even protect against horizontal transfer or retropositioning of their nucleic acids such as TEs.

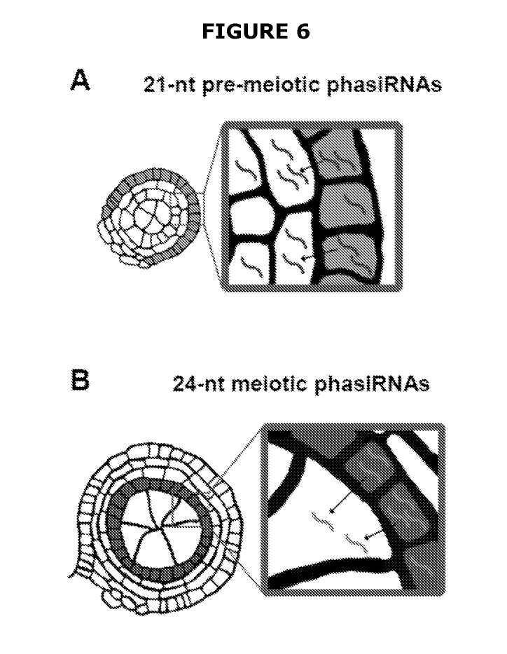

Alternatively, phasiRNAs may serve as mobile signals coordinating anther development. Anthers lack an organizing center, in contrast to the meristem regions of shoots and roots. Meristems organize a continuum of developmental stages displaced from the stem cell population, while anthers “self-organize” tissue layers and the entire organ progresses through development as one unit with high fidelity and temporal regularity. The potential movement of phasiRNAs from the site of biogenesis to neighboring cell layers (FIG. 6) is reminiscent of the TE-derived siRNAs in Arabidopsis pollen, produced in vegetative nuclei and transported into sperm nuclei. Rather than participating in TE silencing, however, phasiRNAs may coordinate cell-type specific expression by an as yet unknown pathway. RDR6 is responsible for the production of both 21-nt and 24-nt phasiRNAs in rice. Interestingly, the RDR6-dependent trans-acting siRNAs in Arabidopsis demonstrated relatively high mobility, further support for the concept that both pre-meiotic and meiotic phasiRNAs could act as mobile signals within developing anthers.

Although both miR2275 and meiotic phasiRNAs have only been reported in grass species, miR2118 is present in dicots. The primary miR2118 targets in dicots are NB-LRR pathogen-defense genes; the 21-nt phasiRNAs produced from the NB-LRR mRNAs function in trans and in cis, and they are expressed constitutively. Therefore, miR2118 and the 21-nt phasiRNAs it triggers have evolved distinct functions in dicot and grass lineages, representing the first case of neofunctionalization among plant miRNAs. One of the two major subgroups with the NB-LRR gene family, the TIR-NB-LRRs, is not found in grass genomes, perhaps hinting at an origin for the miR2118-targeted 21-PHAS precursors. The origin of miR2275 is unknown, but DCL5 is most similar to DCL3, and was earlier named DCL3b. Both miR2275 and DCL5 are absent from dicot genomes, suggesting their recent derivation within the grasses or within related monocots.

Convergent Evolution of Grass phasiRNAs and Mammalian piRNAs

Male reproduction in mammals is also characterized by a high abundance of two classes of small RNAs with accumulation patterns tightly restricted to specific cell types and developmental stages. These small RNAs are known as PIWI-interacting RNAs, or piRNAs. Maize phasiRNAs that we have described and more generally those of grasses share notable similarities with mammalian piRNAs (Table 2), an intriguing case of convergent evolution to produce novel classes of small RNAs in male germinal cells and somatic tissues. PhasiRNAs and mammalian piRNAs both exist in two size classes; the shorter size class occurs pre-meiotically and the longer size accumulates during meiosis. Thus far, neither the grass phasiRNAs nor the majority of mammalian piRNAs have a defined role. This parallelism is an evolutionary puzzle, as is the origin of miR2275, DCL5, and the meiotic phasiRNAs in grasses.

The surprising convergent evolution of small RNAs serving in male reproduction is reminiscent of the separate evolution of imprinting in both the flowering plants and placental mammals. Imprinting differentially marks alleles in gametes by parent-of-origin to set expression after fertilization. Despite the involvement of entirely different tissues in two kingdoms, imprinting accomplishes the same goal of assuring union of male and female gametes to produce the next generation.

What mammals and flowering plants share is a high investment in their progeny. Fertilized embryos are retained within the maternal body and supported by nutritive accessory organs (placenta or endosperm) that do not exist in predecessor taxa. We consider it likely that the piRNAs of mammals and the phasiRNAs of the grasses are contributors to the quality of the male contribution in reproduction, healthy sperm. Despite the fundamental differences between mammalian testes and grass anthers, the parallels in evolving two classes of piRNAs and phasiRNAs, in developmental timing before and during meiosis, the very high abundance, the numerous loci, and lack of obvious mRNA targets suggest that there are considerable evolutionary advantages in each kingdom for these systems for producing small RNAs during male reproduction.

### Experimental Procedures

### Plant Materials

Fertile anthers of the W23 inbred line, mac1 and msca1 introgressed five times into W23, ocl4 in the A188 inbred background, ms23 in the ND101 background, ameiotic1-489 (50% B73+25% A619+25% mixed other or unknown) and am1-praI allele (75% A619+25% mixed other or unknown) were grown in Stanford, Calif. under greenhouse conditions. Anthers were dissected and measured using a micrometer as previously described (Kelliher and Walbot (2011). Dev Biol 350, 32-49).

### RNA Isolation, Library Construction and Illumina Sequencing

Total RNA was isolated using Tri reagent (Molecular Research Center, Cincinnati, Ohio) or Plant RNA Reagent (Invitrogen, Carlsbad, Calif.). Small RNA and RNA-seq libraries were constructed using TruSeq™ Small RNA Sample Prep Kit and TruSeq™ RNA Sample Prep Kit (Illumina, San Diego, Calif.). PARE libraries were constructed as previously described (Zhai et al. (2014). Methods 67, 84-90). All libraries were sequenced on an Illumina Hi-Seq 2000 instrument at the Delaware Biotechnology Institute, Newark, Del.

### Data Handling and Bioinformatics

Ninety-six small RNA libraries were constructed and sequenced, using input materials and generating read counts. Approximately two billion small RNA sequences were obtained after removing adapters and low quality reads, with lengths between 18 and 34 nt. After excluding those matching to structural RNAs (tRNA or rRNA loci), ˜1.5 billion small RNA tags mapped perfectly (no mismatches) back to the reference genome of maize, version AGPv2. Mapping was performed using Bowtie (Langmead et al., 2009). Any read with more than 50 perfect matches (“hits”) to the genome was excluded from further analysis. Abundances of small RNAs in each library were normalized to “TP10M” (transcripts per 10 million) based on the total count of genome-matched reads in that library.

Genome-wide phasing analysis was performed as previously described (Zhai et al. (2011). Genes Dev 25, 2540-2553). To achieve maximum sensitivity, all small RNA libraries were combined to create a union set for detection of the phased distribution of small RNAs. Analysis of phasing was performed in fixed intervals from 19 to 25 nt. Only the 21 and 24 nt intervals generated a result that was significantly higher than background. As a final check of loci with phasing scores higher than or equal to 25, the scores and abundances of small RNAs from each high-scoring locus were graphed and checked visually to remove false positives such as miRNA or unfiltered t/rRNA loci. This yielded 463 loci generating 21-nt pre-meiotic phasiRNAs and 176 loci generating 24-nt meiotic phasiRNAs.

Forty-four RNA-seq libraries were made from 0.4 and 0.7 mm anthers of W23 (wild type), ocl4, and mac1. After trimming RNA-seq reads were mapped to the reference genome using TopHat. Abundances of RNA-seq reads in each library were normalized to TP10M based on the total genome-matched reads of that library.

Five PARE libraries were made. Data analysis and target validation were performed as previously described. In brief, we defined two windows flanking each predicted target site: (1) a small window “W_{S}” of 5 nt (cleavage site ±2 nt), and (2) a large window “W_{L}” of 31 nt (cleavage site ±15 nt). Cleavage sites were filtered to retain only those for which W_{S}/W_{L}≧0.5 in the PARE library in order to remove noisy signals. Target prediction and scoring was done using CleaveLand2.

### In Situ Hybridization

Small RNAs were detected using locked-nucleic acid (LNA) probes synthesized by Exiqon (Woburn, Mass.). Samples were vacuum fixed using 4% paraformaldehyde, and submitted to the histology lab at the A.I. DuPont Hospital for Children (Wilmington, Del.) for paraffin embedding. We followed published protocols for the pre-hybridization, hybridization, post-hybridization, and detection steps.

For PHAS locus and gene transcripts, in situ hybridizations were performed as previously described (Kelliher and Walbot (2014). Plant J 77, 639-652). Probes were synthesized from PCR fragments amplified from genomic DNA followed by transcription using the DIG RNA Labeling Kit (T7/SP6) (Roche, Basel, Switzerland).

### Confocal Microscopy

Confocal images were taken with a Zeiss LSM780 using a C-Apochromat 40× (NA=1.3) oil immersion objective lens at the Delaware Biotechnology Institute, Newark, Del. Sections were excited at 458 nm and auto-fluorescence was detected using a 578 nm-674 nm band pass detector. We also used the same laser for in situ hybridizations, using differential interference contrast (DIC).

Small RNA Detection with Splinted Ligation-Mediated miRNA Detection

miRNAs and phasiRNAs were detected using the USB miRNAtect-It miRNA labeling and detection kit (Affymetrix, Santa Clara Calif.) as previously described (Jeong and Green (2012). Methods 58, 135-143; Jeong et al. (2011). Plant Cell 23, 4185-4207). Each experiment uses 10 μg of total RNA. Analyses were performed.

### Phylogenetic Analysis

Protein sequences of 17 AGOs in maize, 19 in rice and 10 in Arabidopsis were downloaded from NCBI and aligned using MEGA6. The evolutionary history was inferred using the Neighbor-Joining method by MEGA6 and configured by Figtree (http://tree.bio.ed.ac.uk/software/figtree/).

### Example 2

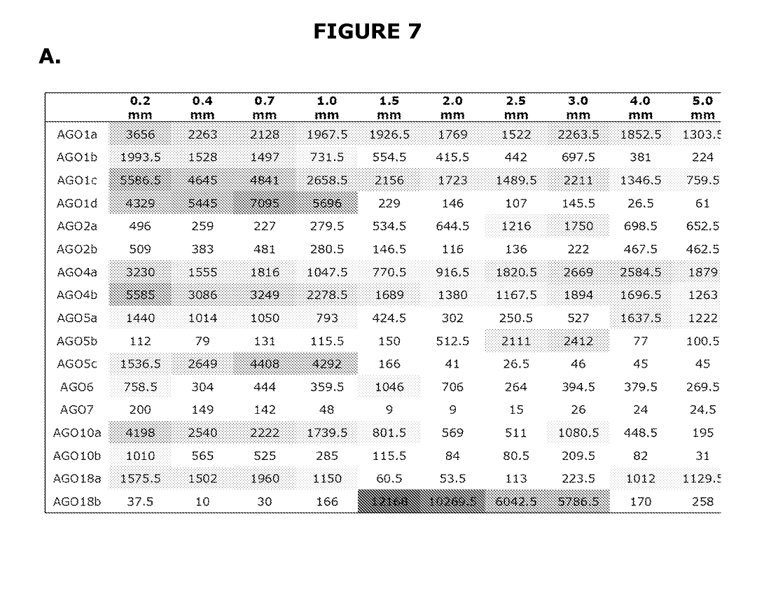

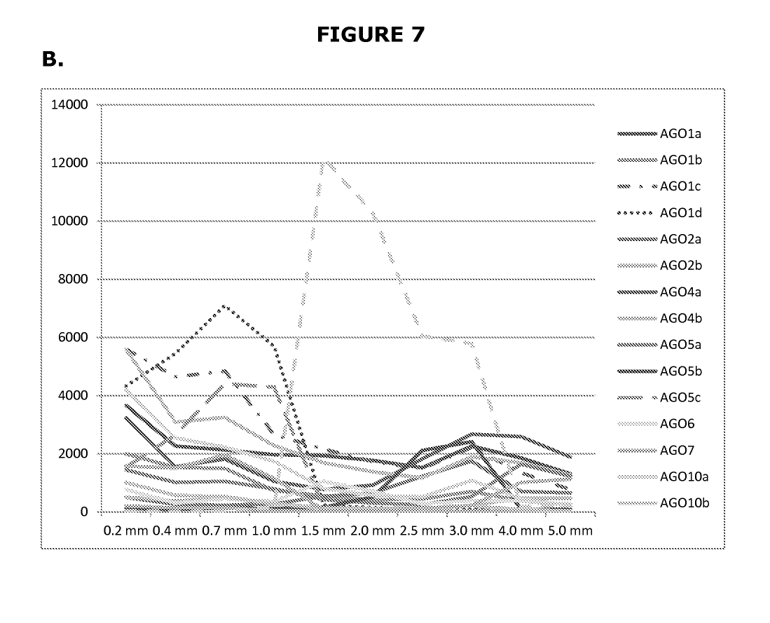

Mutant analyses and particularly targeted genome engineering are used to demonstrate the role of phasiRNAs in grass reproductive biology. Analysis of mRNA transcriptional data for the genes encoding the Argonaute and Dicer proteins has demonstrated enrichment for at least some members of these families, for example, AGO18b, the candidate for binding of the 24-nt phasiRNAs, AGO5c and AGO5b which are highly abundant during meiosis (FIG. 7).

For both maize and rice, CRISPRs are used to specifically knock out AGO18b to critically assess the hypothesis that it is the direct binding partner of 24-nt phasiRNAs, and that the phasiRNA-bound AGO18b protein has an important functional role in male fertility in the grasses. Using the Iowa State University transformation center, over 100 plants are grown with CRISPR short-guide RNAs that target AGO18b. The efficiency of the CRISPR system is high, and characterization of the alleles in the plants will be performed. In the initially transformed generation, both heterozygotes (fertile or partial male sterility) are expected; the latter would demonstrate a role within the pollen grains that inherit a defective allele), or “diallelic” fully AGO18b-deficient lines in which both copies have independently been knocked out resulting in no functional alleles and male sterility.

In addition to the analysis of AGO18b, plants transformed with CRISPR constructs that target DCL5, miR2275a, miR2275b, both “a” and “b” copies of miR2275 together, are regenerated. Knockouts in these genes together with AGO18b target several points in the biogenesis pathway of 24-nt phasiRNAs, providing a multifaceted view of the impact of defects in 24-nt phasiRNA biogenesis. It is believed that all of these lines will be either male sterile or partially male sterile. Based on the expression profile above, it is believe that AGO1d may be a specific triggering factor for 24-nt phasiRNAs, by binding only miR2275 and slicing the 24-nt phasiRNA precursors. This will be tested next with AGO1d-targeting CRISPR constructs.

In addition, starting back in 2012, to prepare materials and begin to assign roles in anther development, numerous AGO, Dicer, RNA polymerase subunits, and other sRNA biogenesis factors that are highly enriched in anthers, or even anther-specific, have been found. For example, UniformMu or RescueMu insertional mutations existed for 30 of these targets represented in 59 lines, with 1 to 7 mutations per target gene. These lines were grown in summer 2012, genotyped to find carriers, and crosses performed to recover families segregating for homozygous “knockout (KO)” mutants. Scoring whether homozygous KOs lack normal gene transcripts and are male-sterile (ms) started in winter 2013 and is continuing. Male sterility identifies a factor as indispensible for normal anther development. By confocal microscopy and analysis, the timing and scope of cellular failure (proliferation or expansion defects, possible cell type-specificity) of each ms case will be pinpointed. It is hypothesized that some 21 and 24 nt phasiRNAs are cell-type specific and hence defects in their generation could result in very specific or unusual sterility phenotypes. Parallel analyses are also conducted in rice using mutants from the large T-DNA populations developed in Taiwan and Korea.

The present invention is not limited to the embodiments described and exemplified above, but is capable of variation and modification within the scope and range of equivalents of the appended claims.

TABLE 1
Coordinates and abundance of the 176 24-phasiRNA-generating loci,
based on summed phasiRNA abundances

| | | | | Total |
|---|---|---|---|---|
| | | | | abundance |
| | | | | (32 fertile |
| | | | | anther |
| 24PHAS_ID | Chr | Start | End | libraries) |
| 24PHAS_NO132 | 4 | 13770958 | 13772199 | 3103289 |
| 24PHAS_NO77 | 2 | 203896086 | 203897904 | 1999863 |
| 24PHAS_NO19 | 1 | 177499181 | 177500787 | 1366722 |
| 24PHAS_NO188 | 5 | 158404577 | 158405146 | 1288822 |
| 24PHAS_NO296 | 10 | 75530023 | 75530974 | 1165521 |
| 24PHAS_NO250 | 8 | 72138181 | 72139229 | 829092 |
| 24PHAS_NO180 | 5 | 157789541 | 157790678 | 801260 |
| 24PHAS_NO187 | 5 | 158403938 | 158404674 | 728806 |
| 24PHAS_NO271 | 9 | 25979840 | 25981416 | 630267 |
| 24PHAS_NO33 | 1 | 183453794 | 183455010 | 588728 |
| 24PHAS_NO15 | 1 | 177416775 | 177417679 | 553720 |
| 24PHAS_NO22 | 1 | 178621235 | 178622594 | 552015 |
| 24PHAS_NO13 | 1 | 176517459 | 176518793 | 542183 |
| 24PHAS_NO16 | 1 | 177422752 | 177423631 | 517141 |
| 24PHAS_NO116 | 4 | 3998971 | 4000258 | 511355 |
| 24PHAS_NO120 | 4 | 6727345 | 6728634 | 510708 |
| 24PHAS_NO258 | 8 | 132726846 | 132728134 | 431810 |
| 24PHAS_NO206 | 6 | 31919056 | 31920345 | 421301 |
| 24PHAS_NO21 | 1 | 177529658 | 177530589 | 382425 |
| 24PHAS_NO82 | 2 | 228312570 | 228313402 | 378050 |
| 24PHAS_NO291 | 10 | 72704862 | 72705767 | 376736 |
| 24PHAS_NO196 | 5 | 211384061 | 211384894 | 355733 |
| 24PHAS_NO115 | 4 | 3911177 | 3912034 | 355698 |
| 24PHAS_NO154 | 4 | 231622669 | 231623718 | 343139 |
| 24PHAS_NO40 | 1 | 183595218 | 183595980 | 339358 |
| 24PHAS_NO128 | 4 | 6881394 | 6882634 | 320416 |
| 24PHAS_NO217 | 6 | 63839816 | 63841393 | 295030 |
| 24PHAS_NO293 | 10 | 75253367 | 75254511 | 279884 |
| 24PHAS_NO126 | 4 | 6837974 | 6838888 | 256169 |
| 24PHAS_NO122 | 4 | 6781451 | 6782343 | 256162 |
| 24PHAS_NO198 | 6 | 30514224 | 30515154 | 242024 |
| 24PHAS_NO127 | 4 | 6844510 | 6845424 | 227460 |
| 24PHAS_NO88 | 2 | 228661326 | 228662445 | 219651 |
| 24PHAS_NO125 | 4 | 6829228 | 6830120 | 216319 |
| 24PHAS_NO123 | 4 | 6797090 | 6797983 | 215803 |
| 24PHAS_NO28 | 1 | 178955728 | 178956490 | 201209 |
| 24PHAS_NO27 | 1 | 178954783 | 178955327 | 199520 |
| 24PHAS_NO23 | 1 | 178626733 | 178628044 | 195941 |
| 24PHAS_NO200 | 6 | 31033415 | 31034533 | 188426 |
| 24PHAS_NO298 | 10 | 75534057 | 75535249 | 187336 |
| 24PHAS_NO173 | 5 | 70575907 | 70577293 | 183572 |
| 24PHAS_NO150 | 4 | 203014019 | 203015069 | 172273 |
| 24PHAS_NO99 | 3 | 157480344 | 157481296 | 163033 |
| 24PHAS_NO31 | 1 | 178966448 | 178967018 | 159558 |
| 24PHAS_NO48 | 1 | 187353324 | 187354706 | 155120 |
| 24PHAS_NO121 | 4 | 6771596 | 6772044 | 152838 |
| 24PHAS_NO213 | 6 | 62927027 | 62928458 | 152628 |
| 24PHAS_NO11 | 1 | 176488245 | 176489869 | 148034 |
| 24PHAS_NO262 | 9 | 12697058 | 12697899 | 142864 |
| 24PHAS_NO108 | 4 | 2809993 | 2810803 | 136488 |
| 24PHAS_NO112 | 4 | 3780146 | 3781052 | 133090 |
| 24PHAS_NO20 | 1 | 177506123 | 177506619 | 127230 |
| 24PHAS_NO270 | 9 | 24952056 | 24953128 | 125476 |
| 24PHAS_NO183 | 5 | 158040150 | 158040934 | 121304 |
| 24PHAS_NO24 | 1 | 178691162 | 178692320 | 120895 |
| 24PHAS_NO185 | 5 | 158301218 | 158302442 | 120158 |
| 24PHAS_NO215 | 6 | 63260796 | 63261632 | 119306 |
| 24PHAS_NO294 | 10 | 75518504 | 75519695 | 118159 |
| 24PHAS_NO18 | 1 | 177496500 | 177497261 | 115858 |
| 24PHAS_NO210 | 6 | 32378820 | 32380157 | 115550 |
| 24PHAS_NO81 | 2 | 228309922 | 228310825 | 114850 |
| 24PHAS_NO143 | 4 | 135894713 | 135896577 | 114591 |
| 24PHAS_NO86 | 2 | 228423636 | 228424851 | 113414 |
| 24PHAS_NO288 | 10 | 1781835 | 1782717 | 104903 |
| 24PHAS_NO84 | 2 | 228422136 | 228422801 | 102721 |
| 24PHAS_NO109 | 4 | 2812206 | 2813663 | 99331 |
| 24PHAS_NO285 | 9 | 150140202 | 150141664 | 96207 |
| 24PHAS_NO236 | 7 | 18043244 | 18044126 | 95068 |
| 24PHAS_NO133 | 4 | 13851524 | 13853243 | 92622 |
| 24PHAS_NO89 | 2 | 228663332 | 228664019 | 86712 |
| 24PHAS_NO25 | 1 | 178747100 | 178748229 | 86187 |
| 24PHAS_NO29 | 1 | 178960922 | 178961492 | 85165 |
| 24PHAS_NO174 | 5 | 70579428 | 70580237 | 83239 |
| 24PHAS_NO267 | 9 | 24369311 | 24370552 | 82608 |
| 24PHAS_NO282 | 9 | 96938506 | 96939004 | 82607 |
| 24PHAS_NO83 | 2 | 228362599 | 228363384 | 82091 |
| 24PHAS_NO208 | 6 | 32276828 | 32277252 | 81439 |
| 24PHAS_NO219 | 6 | 63880637 | 63881518 | 80144 |
| 24PHAS_NO237 | 7 | 18045236 | 18046556 | 79158 |
| 24PHAS_NO6 | 1 | 23832371 | 23833166 | 75324 |
| 24PHAS_NO124 | 4 | 6803596 | 6804391 | 75324 |
| 24PHAS_NO161 | 5 | 1179957 | 1181246 | 71026 |
| 24PHAS_NO134 | 4 | 13864166 | 13865840 | 70478 |
| 24PHAS_NO207 | 6 | 32276327 | 32276848 | 66555 |
| 24PHAS_NO283 | 9 | 96994409 | 96995361 | 66368 |
| 24PHAS_NO172 | 5 | 70197919 | 70198777 | 57188 |
| 24PHAS_NO286 | 10 | 1775462 | 1776584 | 56816 |
| 24PHAS_NO214 | 6 | 63144897 | 63146231 | 55849 |
| 24PHAS_NO257 | 8 | 132598963 | 132599892 | 53149 |
| 24PHAS_NO87 | 2 | 228612730 | 228613395 | 53074 |
| 24PHAS_NO307 | 10 | 145813714 | 145814811 | 52153 |
| 24PHAS_NO268 | 9 | 24453420 | 24454491 | 49581 |
| 24PHAS_NO17 | 1 | 177487500 | 177488188 | 49017 |
| 24PHAS_NO159 | 5 | 1177705 | 1178753 | 46929 |
| 24PHAS_NO35 | 1 | 183562643 | 183563909 | 43514 |
| 24PHAS_NO42 | 1 | 183674806 | 183676238 | 42658 |
| 24PHAS_NO14 | 1 | 176755600 | 176756505 | 42633 |
| 24PHAS_NO39 | 1 | 183565888 | 183566698 | 40962 |
| 24PHAS_NO5 | 1 | 19441411 | 19442196 | 39546 |
| 24PHAS_NO211 | 6 | 32390856 | 32391306 | 39296 |
| 24PHAS_NO45 | 1 | 183686510 | 183687294 | 37142 |
| 24PHAS_NO158 | 5 | 1176522 | 1177616 | 36290 |
| 24PHAS_NO160 | 5 | 1178892 | 1179819 | 33688 |
| 24PHAS_NO246 | 8 | 3691249 | 3692129 | 33000 |
| 24PHAS_NO26 | 1 | 178836111 | 178836945 | 32505 |
| 24PHAS_NO178 | 5 | 157648306 | 157648947 | 31435 |
| 24PHAS_NO209 | 6 | 32279981 | 32280790 | 30552 |
| 24PHAS_NO201 | 6 | 31056992 | 31058235 | 30381 |
| 24PHAS_NO43 | 1 | 183678460 | 183679125 | 29215 |
| 24PHAS_NO47 | 1 | 183756201 | 183757010 | 28374 |
| 24PHAS_NO157 | 5 | 1175530 | 1176290 | 27624 |
| 24PHAS_NO202 | 6 | 31122577 | 31123627 | 27587 |
| 24PHAS_NO114 | 4 | 3910139 | 3910994 | 26890 |
| 24PHAS_NO117 | 4 | 4051034 | 4052178 | 21695 |
| 24PHAS_NO111 | 4 | 3762598 | 3763911 | 21279 |
| 24PHAS_NO100 | 3 | 160451658 | 160452948 | 21277 |
| 24PHAS_NO203 | 6 | 31164646 | 31165982 | 20389 |
| 24PHAS_NO218 | 6 | 63876976 | 63877880 | 19598 |
| 24PHAS_NO113 | 4 | 3785579 | 3786267 | 18843 |
| 24PHAS_NO186 | 5 | 158403451 | 158403851 | 18781 |
| 24PHAS_NO156 | 5 | 1139186 | 1139587 | 18084 |
| 24PHAS_NO92 | 3 | 48326082 | 48327275 | 17978 |
| 24PHAS_NO10 | 1 | 176408191 | 176408711 | 15475 |
| 24PHAS_NO197 | 6 | 30513432 | 30514143 | 14994 |
| 24PHAS_NO181 | 5 | 158039310 | 158039806 | 14698 |
| 24PHAS_NO44 | 1 | 183681377 | 183683436 | 14353 |
| 24PHAS_NO179 | 5 | 157789005 | 157789405 | 13290 |
| 24PHAS_NO245 | 8 | 3673291 | 3674412 | 12239 |
| 24PHAS_NO199 | 6 | 30986176 | 30987417 | 8283 |
| 24PHAS_NO37 | 1 | 183564180 | 183564725 | 8168 |
| 24PHAS_NO216 | 6 | 63830820 | 63831244 | 7743 |
| 24PHAS_NO144 | 4 | 140349656 | 140350273 | 7087 |
| 24PHAS_NO204 | 6 | 31295471 | 31296545 | 7067 |
| 24PHAS_NO259 | 8 | 156768362 | 156768834 | 6737 |
| 24PHAS_NO205 | 6 | 31410609 | 31411441 | 5644 |
| 24PHAS_NO264 | 9 | 21732903 | 21733565 | 4696 |
| 24PHAS_NO107 | 4 | 2809057 | 2809938 | 4249 |
| 24PHAS_NO272 | 9 | 26362364 | 26363076 | 3588 |
| 24PHAS_NO36 | 1 | 183563860 | 183564260 | 3456 |
| 24PHAS_NO153 | 4 | 231610010 | 231610411 | 2875 |
| 24PHAS_NO46 | 1 | 183688098 | 183688546 | 2629 |
| 24PHAS_NO292 | 10 | 75179896 | 75180920 | 2507 |
| 24PHAS_NO171 | 5 | 70184569 | 70185185 | 2162 |
| 24PHAS_NO273 | 9 | 26391377 | 26391968 | 2161 |
| 24PHAS_NO162 | 5 | 1181362 | 1181929 | 1782 |
| 24PHAS_NO166 | 5 | 32090166 | 32090566 | 1758 |
| 24PHAS_NO63 | 2 | 36837216 | 36837616 | 1699 |
| 24PHAS_NO212 | 6 | 32400293 | 32401152 | 1631 |
| 24PHAS_NO97 | 3 | 143696807 | 143697710 | 1463 |
| 24PHAS_NO287 | 10 | 1776519 | 1777183 | 1085 |
| 24PHAS_NO4 | 1 | 19440812 | 19441260 | 1050 |
| 24PHAS_NO54 | 1 | 245219118 | 245219663 | 901 |
| 24PHAS_NO251 | 8 | 72139212 | 72139612 | 775 |
| 24PHAS_NO34 | 1 | 183455066 | 183455874 | 650 |
| 24PHAS_NO184 | 5 | 158211375 | 158212960 | 517 |
| 24PHAS_NO155 | 4 | 231666282 | 231667210 | 481 |
| 24PHAS_NO191 | 5 | 195273170 | 195273570 | 447 |
| 24PHAS_NO41 | 1 | 183596130 | 183596531 | 397 |
| 24PHAS_NO8 | 1 | 106668445 | 106668918 | 246 |
| 24PHAS_NO106 | 3 | 223741305 | 223741826 | 246 |
| 24PHAS_NO102 | 3 | 167473244 | 167473644 | 239 |
| 24PHAS_NO229 | 6 | 122327004 | 122327572 | 227 |
| 24PHAS_NO297 | 10 | 75531054 | 75531526 | 163 |
| 24PHAS_NO105 | 3 | 220283559 | 220284198 | 138 |
| 24PHAS_NO90 | 3 | 2906577 | 2907051 | 108 |
| 24PHAS_NO269 | 9 | 24456512 | 24457104 | 86 |
| 24PHAS_NO61 | 2 | 8270833 | 8271233 | 83 |
| 24PHAS_NO49 | 1 | 195393404 | 195393948 | 67 |
| 24PHAS_NO71 | 2 | 77065564 | 77066107 | 67 |
| 24PHAS_NO256 | 8 | 132367767 | 132368167 | 65 |
| 24PHAS_NO78 | 2 | 211175568 | 211175993 | 59 |
| 24PHAS_NO12 | 1 | 176506993 | 176508019 | 58 |
| 24PHAS_NO76 | 2 | 184176846 | 184177317 | 54 |
| 24PHAS_NO240 | 7 | 91724328 | 91724776 | 46 |
| 24PHAS_NO295 | 10 | 75519751 | 75520151 | 44 |
| 24PHAS_NO242 | 7 | 107811706 | 107812130 | 22 |

TABLE 2
Parallels between grass phasiRNAs and mammalian piRNAs.

| | Grass (Maize) phasiRNA* | | Mammalian piRNA^{†} | |
|---|---|---|---|---|
| Organ | Anther | | Testis | |
| Developmental | Pre-meiotic | Meiotic | Pre- | Pachytene |
| stage | | | pachytene | |
| Peak timing | 0.4 mm | 2.0 mm | 12.5 dpp | 17.5 dpp |
| | anther | anther | | |
| Size | 21-nt | 24-nt | 26~27-nt | 29~30-nt |
| AGO partner | AGO5c^{‡} | AGO18b^{‡} | MILI, | MIWI |
| | | | MIWI2 | |
| Master regulator | miR2118 | miR2275 | Unknown | A-myb |
| Number of loci | 463 | 176 | ~900 | ~100 |

| Abundance | Very high | |
|---|---|---|
| Distribution | Present on all chromosomes; most loci are clustered | |
| Precursor | Non-coding, Pol II transcripts | |
| Single copy? | Nearly all | |
| Repeat | Few | |
| associated? | | |
| Impacts on | Mis-regulated in sterile mutants; | |
| fertility | biogenesis defects can cause sterility | |
| Targeting | The majority lack complementarity to TEs or other loci | |
| Function(s) | Unknown | Mainly unknown; |
| | | a subset silence TEs |

*Properties of phasiRNAs are from this study and prior reports.
^{†}Mammalian piRNAs have been extensively characterized.
^{‡}Proposed binding partners

What is claimed:

1. A method for controlling male fertility of a plant, comprising regulating a biological activity of a phasiRNA in a male reproductive organ of the plant, wherein the phasiRNA is selected from the group consisting of 21-nt phasiRNAs and 24-nt phasiRNAs, whereby the male fertility of the plant is increased or decreased.

2. The method of claim 1, further comprising regulating the expression of the phasiRNA in cells of the male reproductive organ, whereby the biological activity of the phasiRNA is increased or decreased.

3. The method of claim 2, further comprising regulating the expression in cells of the male reproductive organ of an mRNA precursor (PHAS) of the phasiRNA, a 22-nt microRNA (miRNA) capable of cleaving the PHAS to make the phasiRNA, or a facilitating protein capable of regulating the expression of the phasiRNA in the plant, whereby the expression of the phasiRNA is increased or decreased.

4. The method of claim 3, further comprising introducing into cells of the male reproductive organ an effective amount of a nucleic acid molecule that is antagonistic to the phasiRNA, the mRNA precursor (PHAS), or the 22-nt microRNA (miRNA), whereby the expression of the phasiRNA, the mRNA precursor (PHAS), or the 22-nt microRNA (miRNA) is increased or decreased.

5. The method of claim 3, further comprising regulating the expression of RNA-Dependent RNA Polymerase 6 (RDR6) in cells of the male reproductive organ, whereby the expression of the mRNA precursor (PHAS) is increased or decreased.

6. The method of claim 3, wherein the phasiRNA is a 21-nt phasiRNA, the 22-nt miRNA is miR2118, and the facilitating protein is selected from the group consisting a dicer protein and an Argonaute (AGO) protein.

7. The method of claim 6, wherein the dicer protein is DICER-LIKE4 (DCL4).

8. The method of claim 6, wherein the Argonaute (AGO) protein is an AGO5-related protein.

9. The method of claim 8, wherein the plant is rice and the AGO5-related protein is Meiosis Arrested At Leptotene 1 (MEL1).

10. The method of claim 3, wherein the phasiRNA is a 24-nt phasiRNA, the 22-nt miRNA is miR2275, and the facilitating protein is selected from the group consisting a dicer protein and an Argonaute (AGO) protein.

11. The method of claim 10, wherein the dicer protein is DICER-LIKE5 (DCL5).

12. The method of claim 10, wherein the Argonaute (AGO) protein is an AGO18 protein.

13. The method of claim 12, wherein the plant is maize and the AGO18 protein is selected from the group consisting of GRMZM2G105250 and GRMZM2G457370.

14. The method of claim 1, wherein the plant is a monocotyledon.

15. The method of claim 1, wherein the plant is maize.

16. The method of claim 1, whereby the plant is male sterile.

17. A male sterile plant obtained in accordance with claim 16.

18. A plant cell or tissue obtained from the male sterile plant of claim 17.

19. A method for producing a hybrid seed, comprising crossing the male sterile plant of claim 17 with another plant, whereby a hybrid seed is produced.

20. The hybrid seed produced in accordance with claim 19.
